(19) United States
(12) Patent Application Publication
(10) Pub. No.: US 2023/0113761 A1
(43) Pub. Date: Apr. 13, 2023

(54) METHOD AND COMPOSITION FOR MIMICKING BIOLOGICAL BENEFITS OF CALORIC RESTRICTION BY ADMINISTRATION OF B-AMINOISOBUTYRIC ACID

(71) Applicant: NANJING NUTRABUILDING BIO-TECH CO., LTD., Nanjing (CN)

(72) Inventors: Mingru WANG, Nanjing (CN); Ronghua YI, Nanjing (CN); Kylin LIAO, Nanjing (CN)

(21) Appl. No.: 17/959,466

(22) Filed: Oct. 4, 2022

(30) Foreign Application Priority Data
Oct. 8, 2021 (WO) PCT/CN2021/122677

(51) Int. Cl. A61K 31/197; A61P 3/04

(52) U.S. Cl. CPC A61K 31/197; A61P 3/04

(57) ABSTRACT

Among others, the present invention provides methods and compositions for mimicking one or more biological benefits of caloric restriction in a mammal, including administrating to the mammal an effective amount of β-aminoisobutyric acid, an analog, metabolite or derivative thereof, or a pharmaceutically acceptable salt, ester, polymer, acid thereof.

### CROSS REFERENCE TO RELATED APPLICATION

This application claims the benefit of a PCT International Application Number PCT/CN2021/122677, filed on Oct. 08, 2021, the content of which is hereby incorporated by reference in its entirety for all purposes.

### FIELD OF THE INVENTION

This invention generally relates to the field of methods for mimicking caloric restriction in a mammal, and more specifically, relates to methods and compositions for mimicking the biological benefits of caloric restriction in a mammal, involving administrating to the mammal an effective amount of β-aminoisobutyric acid, an analog, metabolite or derivative thereof, or a pharmaceutically acceptable salt, ester, polymer, acid thereof.

### BACKGROUND OF THE INVENTION

Caloric restriction (CR), also known as dietary restriction, is the reduction of food intake without malnutrition. Studies in numerous species have demonstrated that reduction of calories 30-50% below ad libitum levels of a nutritious diet can increase lifespan, reduce the incidence and delay the onset of age-related diseases, improve stress resistance, and decelerate functional decline (Aging Cell. 2006 Apr;5(2):97-108). There are various calorie-restricted diets. The general principle is to eat low-calorie foods, such as vegetables and fruits, and to avoid eating some higher-calorie foods, such as starches and fatty meats. Long-term calorie-restricted diets, if not carefully designed, will likely lead to malnutrition and health hazards. And not eating carbohydrates for a long time will affect the mood and may lead to depression in severe cases. Even if the caloric restriction is beneficial to lifespan and health, it is difficult for most people to implement such restrictions in our lives for social, economic and medical reasons, especially, in a long term. To overcome the difficulties, more and more researches focused on developing medicines to mimic the beneficial effects of caloric restriction without actually restricting caloric intake. Such medicines are known as caloric restriction mimetics (CRM).

An effective caloric restriction mimetic (CRM) is a pharmaceutical or natural compound which would alter the key metabolic pathways involved in the effects of caloric restriction itself, therefore reproduce one or more principal biological effects of caloric restriction without reducing food intake, which may be especially suitable for mid- to late-life periods. Many putative calorie restriction mimetics have been found potentially useful in humans. For example, drugs that inhibit glycolysis (2-deoxyglucose), enhance insulin action (metformin), or affect stress signaling pathways (resveratrol), are being assessed as caloric restriction mimetics (Aging Cell. 2006 Apr;5(2):97-108). Promising results have emerged from initial studies regarding physiological responses which resemble those observed in caloric restriction. Ultimately, lifespan analysis and expanded toxicity studies must be accomplished to fully assess the potential of any caloric restriction mimetics. Nonetheless, this strategy offers a very promising and expanding research endeavor.

β-aminoisobutyric acid (BAIBA) is a non-protein amino acid, which is released by skeletal muscle through a proliferator-activated receptor-gamma coactivator-1a (PGC-1a)-dependent pathway during physical activity, and has been discovered as a novel endogenous protective myokine, regulating adipose tissue browning, improving insulin sensitivity and protecting against a high-fat diet-induced obesity. There are two enantiomers of BAIBA in biological systems: D-BAIBA and L-BAIBA. L-BAIBA is generated from catabolic reactions of branched-chain amino acid L-valine. Specifically, L-BAIBA is produced by the mitochondrial enzyme 4-aminobutyrate aminotransferase (ABAT) in the transaminase reaction between the downstream metabolite of L-valine L-methyl-malonyl semialdehyde (L-MMS) and L-glutamate. D-BAIBA is produced in the cytosol from thymine in a metabolic pathway involving dihydropyrimidine dehydrogenase (DPYD), dihydropyrimidinase (DPYS), and β-ureidopropionase (UPB1) and is further metabolized in mitochondria by alanine:glyoxylate aminotransferase 2 (AGXT2) to D-methylmalonate semialdehyde (D-MMS) (Nutrients. 2019 Mar; 11(3): 524).

In the present invention, we have discovered a novel therapeutic application for β-aminoisobutyric acid as an effective caloric restriction mimetic that shows health benefits.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

This invention generally relates to compounds, compositions and methods for mimicking one or more biological benefits of caloric restriction in a mammal, comprising administrating to the mammal an effective amount of β-aminoisobutyric acid, an analog, metabolite, or derivative thereof, or a pharmaceutically acceptable salt, acid, ester, polymer thereof. According to the present invention, it was surprisingly found that β-aminoisobutyric acid can function as a caloric restriction mimic, extending lifespan and providing other health benefits. It is believed that this invention is the first time to propose and conduct β-aminoisobutyric acid as a caloric restriction mimic.

One aspect of the present invention provides a method for mimicking one or more biological benefits of caloric restriction in a mammal, comprising administrating to the mammal an effective amount of β-aminoisobutyric acid, or an analog, metabolite or derivative thereof, or a pharmaceutically acceptable salt, acid, ester, polymer thereof.

In some embodiments, the one or more biological benefits comprise: lowering heart rate, blood pressure, low-density lipoprotein, cholesterol and triglycerides; raising high-density lipoprotein; improving insulin sensitivity and normalizing blood glucose; maintaining DNA integrity; reducing oxidative stress; decreasing body temperature; reducing body fat mass, including visceral obesity, while increasing muscle mass; increasing lean mass; improving body composition; losing weight; enhancing the ability to engage in sports activities; improving brain function, including memory, cognition, and mood; or stimulating growth factors.

In some embodiments, β-aminoisobutyric acid is of configuration L or D or a form of a mixture of L and D configurations.

In some embodiments, β-aminoisobutyric acid is administrated in an amount ranging from 0.1 mg/day-5000 mg/day.

In some embodiments, the β-aminoisobutyric acid is administrated in an amount ranging from 100 mg/day-2500 mg/day. In some embodiments, β-aminoisobutyric acid is administrated in an amount ranging from 1 mg/day-4000 mg/day, 10 mg/day-3000 mg/day, 100 mg/day-2500 mg/day, 300 mg/day-2000 mg/day, 500 mg/day-1800 mg/day, or 700-1500 mg/day.

In some embodiments, the β-aminoisobutyric acid is administrated in a form of aqueous solution, aqueous suspension, capsule, drop, granule, liquid, powder, syrup, tablet, functionalized food, beverage, toothpaste, or sublingual articles.

In some embodiments, the mammal is human.

In some embodiments, the β-aminoisobutyric acid is administrated orally, by intravenous injection, by intramuscular injection, intraperitoneally or sublingually.

In some embodiments, the β-aminoisobutyric acid is prepared in a form of nutritional, drinking, or pharmaceutical composition, for use in a food, drink, nutritional, or pharmaceutical products.

In some embodiments, the β-aminoisobutyric acid is administrated as a dietary supplement or an ingredient in a food.

Another aspect of this invention relates to a composition capable of mimicking one or more biological benefits of caloric restriction in a mammal, comprising an effective amount of β-aminoisobutyric acid, an analog, metabolite or derivative thereof, or a pharmaceutically acceptable salt, ester, polymer, acid thereof.

In some embodiments, the one or more biological benefits comprise: lowering heart rate, blood pressure, low-density lipoprotein, cholesterol and triglycerides; raising high-density lipoprotein; improving insulin sensitivity and normalizing blood glucose; maintaining DNA integrity; reducing oxidative stress; decreasing body temperature; reducing body fat mass, including visceral obesity, while increasing muscle mass; increasing lean mass; improving body composition; losing weight; enhancing the ability to engage in sports activities; improving brain function, including memory, cognition, and mood; or stimulating growth factors.

In some embodiments, β-aminoisobutyric acid is of configuration L or D or a form of a mixture of L and D configurations.

In some embodiments, β-aminoisobutyric acid is administrated in an amount ranging from 0.1 mg/day-5000 mg/day.

In some embodiments, the β-aminoisobutyric acid is administrated in an amount ranging from 100 mg/day-2500 mg/day. In some embodiments, β-aminoisobutyric acid is administrated in an amount ranging from 1 mg/day-4000 mg/day, 10 mg/day-3000 mg/day, 100 mg/day-2500 mg/day, 300 mg/day-2000 mg/day, 500 mg/day-1800 mg/day, or 700-1500 mg/day.

In some embodiments, the β-aminoisobutyric acid is administrated in a form of aqueous solution, aqueous suspension, capsule, drop, granule, liquid, powder, syrup, tablet, functionalized food, beverage, toothpaste, or sublingual articles.

In some embodiments, the mammal is human.

In some embodiments, the β-aminoisobutyric acid is administrated orally, by intravenous injection, by intramuscular injection, intraperitoneally or sublingually.

In some embodiments, the β-aminoisobutyric acid is prepared in a form of nutritional, drinking, or pharmaceutical composition, for use in a food, drink, nutritional, or pharmaceutical products.

In some embodiments, the β-aminoisobutyric acid is administrated as a dietary supplement or an ingredient in a food.

In some embodiments, the composition is a dietary composition or supplement.

A further aspect of the invention relates to use of β-aminoisobutyric acid in manufacturing a composition capable of mimicking one or more biological benefits of caloric restriction in a mammal.

In some embodiments, the biological benefits comprise: lowering heart rate, blood pressure, low-density lipoprotein, cholesterol and triglycerides; raising high-density lipoprotein; improving insulin sensitivity and normalizing blood glucose; maintaining DNA integrity; reducing oxidative stress; decreasing body temperature; reducing body fat mass, including visceral obesity, while increasing muscle mass; increasing lean mass; improving body composition; losing weight; enhancing the ability to engage in sports activities; improving brain function, including memory, cognition, and mood; or stimulating growth factors.

In some embodiments, β-aminoisobutyric acid is of configuration L or D or a form of a mixture of L and D configurations.

In some embodiments, β-aminoisobutyric acid is administrated in an amount ranging from 0.1 mg/day-5000 mg/day.

In some embodiments, the β-aminoisobutyric acid is administrated in an amount ranging from 100 mg/day-2500 mg/day. In some embodiments, β-aminoisobutyric acid is administrated in an amount ranging from 1 mg/day-4000 mg/day, 10 mg/day-3000 mg/day, 100 mg/day-2500 mg/day, 300 mg/day-2000 mg/day, 500 mg/day-1800 mg/day, or 700-1500 mg/day.

In some embodiments, the β-aminoisobutyric acid is administrated in a form of aqueous solution, aqueous suspension, capsule, drop, granule, liquid, powder, syrup, tablet, functionalized food, beverage, toothpaste, or sublingual articles.

In some embodiments, the mammal is human.

In some embodiments, the β-aminoisobutyric acid is administrated orally, by intravenous injection, by intramuscular injection, intraperitoneally or sublingually.

In some embodiments, the β-aminoisobutyric acid is prepared in a form of nutritional, drinking, or pharmaceutical composition, for use in a food, drink, nutritional, or pharmaceutical products.

In some embodiments, the β-aminoisobutyric acid is administrated as a dietary supplement or an ingredient in a food.

### BRIEF DESCRIPTION OF THE DRAWINGS

FIG. 1 is a graph of the body weight changes of four groups of mice during 8 weeks of feeding intervention.

FIG. 2 is a graph of the fat mass changes of four groups of mice during 8 weeks of feeding intervention.

FIG. 3 is a graph of the lean mass changes of four groups of mice during 8 weeks of feeding intervention.

FIG. 4 is a graph of the free water changes of four groups of mice during 8 weeks of feeding intervention.

FIG. 5 is a graph comparing total cholesterol (TCHO) levels of four groups of mice measured after 8 weeks of treatment.

FIG. 6 is a graph comparing triglyceride (TG) levels of four groups of mice measured after 8 weeks of treatment.

FIG. 7 is a graph comparing low-density lipoprotein (LDL) levels of four groups of mice measured after 8 weeks of treatment.

FIG. 8 is a graph comparing high-density lipoprotein (HDL) levels of four groups of mice measured after 8 weeks of treatment.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

Generally speaking, various embodiments of the present invention provide for compositions and methods of mimicking one or more biological benefits of caloric restriction in a mammal, comprising administrating to the mammal an effective amount of β-aminoisobutyric acid, an analog, metabolite or derivative thereof, or a pharmaceutically acceptable salt, ester, polymer, acid thereof. Particularly, β-aminoisobutyric acid can be used as a caloric restriction mimic. Moreover, β-aminoisobutyric acid is administrated in a variety of forms, such as aqueous solution, aqueous suspension, capsule, drop, granule, liquid, powder, syrup, tablet, functionalized food, beverage, toothpaste, or sublingual articles.

### Definitions

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed combinations, compositions or kits, or be a recipient of the disclosed methods. Although the animal subject is preferably a human, the methods and compositions of the invention have application in veterinary medicine as well, e.g., for the treatment of domesticated species such as canine, feline, murine, and various other pets; farm animal species such as bovine, equine, ovine, caprine, porcine, etc.; and wild animals, e.g., in the wild or in a zoological garden, such as non-human primates.

As used herein, the term “administration” refers to the process of delivering a disclosed combination, composition or kit to a subject. The combination, compositions or kits can be administered in a variety of ways, including orally, intragastrically, and parenterally (e.g., intravenous and intraarterial as well as other suitable parenteral routes), and the like.

As used herein, the term “effective amount” refers to an amount that is required to achieve the effect as taught herein. An effective amount herein includes, but is not limited to, the amount necessary to mimick biological benefits of caloric restriction in a mammal; and/or the amount necessary to lower heart rate, blood pressure, low-density lipoprotein, cholesterol and triglycerides; to raise high-density lipoprotein; to improve insulin sensitivity and normalize blood glucose; to maintain DNA integrity; to reduce oxidative stress; to decrease body temperature; to reduce body fat mass, including visceral obesity, while increasing muscle mass; to increase lean mass; to improve body composition; to lose weight; to enhance the ability to engage in sports activities; to improve brain function, including memory, cognition, and mood; or to stimulate growth factors. In accordance with the present disclosure, a suitable single dose size is that which, when administered one or more times over a suitable period of time, achieves the above-described effects.

As used herein, the term “pharmaceutically acceptable” means pharmaceutically, physiologically, alimentarily, or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “caloric restriction (CR)” and the like, refers to the dietary manipulations in yeast, worms, flies, rodents and other mammals, including humans, that result in increased life span and other biological benefits through reduction of caloric intake.

As used herein, the term “calorie restriction mimic” or “calorie restriction mimetic”, and the like, is pharmaceutical or natural compound or substance, mixture of compounds or substances that recapitulate calorie restriction conditions without the usual requirement for a reduction in calorie content of the diet. With extended use, calorie restriction mimics up- and down-regulate gene expression and cellular proteins to resemble those associated with calorie-restricted profiles as well as to decrease insulin resistance (lower fasting blood glucose levels) and increase glucose uptake, also similar to changes seen in calorie restriction.

β-Aminoisobutyric acid may be used directly as active agent or may be generated in vivo after administration of a prodrug or analog or derivative thereof, or one of its intermediate metabolites. As used herein, the term “metabolite” is considered to be any substance resulting from the metabolism of β-Aminoisobutyric acid. The term “derivatives” include inorganic or organic salts, esters or amides of β-aminoisobutyric acid. The terminal carboxylic group of β-aminoisobutyric acid may be in particular under the form of an ester, for example lower alkyl ester, or of an amide. The term “prodrug” is dedicated to refer to any substance that gives rise to a pharmacologically active form of BAIBA although not itself active. In particular, the active agent is β-aminoisobutyric acid. It may be of L (i.e., S) or D (i.e., R) configuration or a mixture of L and D configurations.

The following examples are illustrative of select embodiments of the present invention and are not meant to limit the scope of the invention.

### EXAMPLES

### Example 1

Mice were divided into groups and named after the diet they consumed. Group A mice were fed ad libitum (control), Group B mice were on high-fat diet (HFD), Group C mice were fed restricted amount of the HFD diet to achieve caloric restriction (HFD+CR), and Group D mice were fed high-fat diet and supplemented with 150 mg/kg β-aminoisobutyric acid (HFD+BAIBA). All groups of mice were housed individually, and fed for months of interventions and had free access to water. Health status was monitored during the intervention.

The four groups of mice were fed for eight weeks, body weight was measured weekly, and body fat mass, lean mass and free water mass were also measured by Body Composition Analyzer to monitor body composition every week. At the end of 8 weeks of treatment, blood was collected for each group of mice, and parameters such as total cholesterol (TCHO), triglyceride (TG), low-density lipoprotein (LDL) and high-density lipoprotein (HDL) were detected. Data were collected and subject to statistical analysis.

FIG. 1 is a graph of the body weight changes of four groups of mice during 8 weeks of feeding intervention. As shown in FIG. 1, the weight of mice in group B (HFD) was significantly greater than that in group A (control); group C (HFD+CR) achieved calorie restriction through a restricted HFD diet, which could significantly reduce body weight compared with group B (HFD), but the way of calorie restriction is likely to lead to malnutrition and cause health risks; the weight of group D (HFD+BAIBA) was also significantly lower than that of group B (HFD), indicating that BAIBA supplementation while feeding a high-fat diet can mimic the weight loss effect of caloric restriction without causing malnutrition.

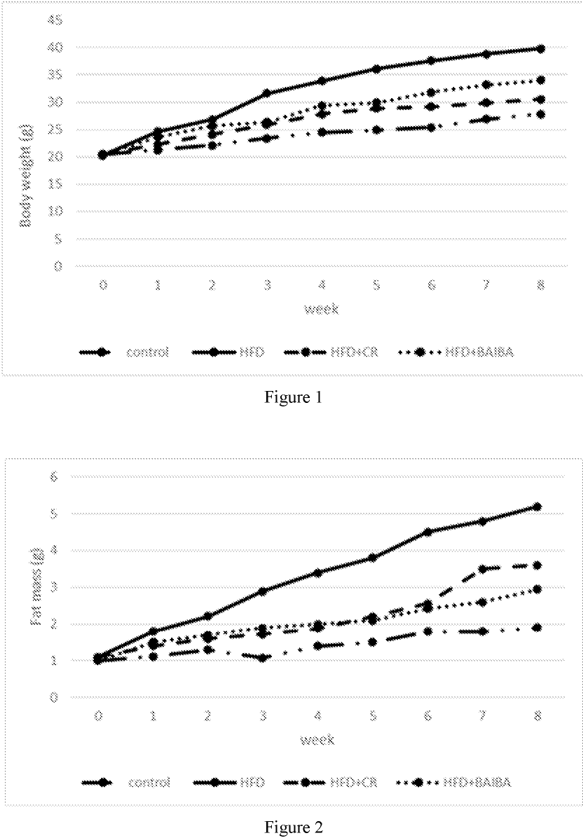

FIG. 2 is a graph of the fat mass changes of four groups of mice during 8 weeks of feeding intervention. As shown in FIG. 2, the fat mass of mice in group B (HFD) was significantly greater than that in group A (control); group C (HFD+CR) achieved calorie restriction through a restricted HFD diet, which could reduce fat mass compared with group B (HFD), but the way of calorie restriction is likely to lead to malnutrition and cause health risks; the fat mass of group D (HFD+BAIBA) was also decreased compared to group B (HFD), and from week 5 of feeding intervention, group D (HFD+BAIBA) even had less fat mass than group C (HFD+CR), indicating that BAIBA supplementation while feeding a high-fat diet can mimic the body fat mass reduction effect of caloric restriction without causing malnutrition.

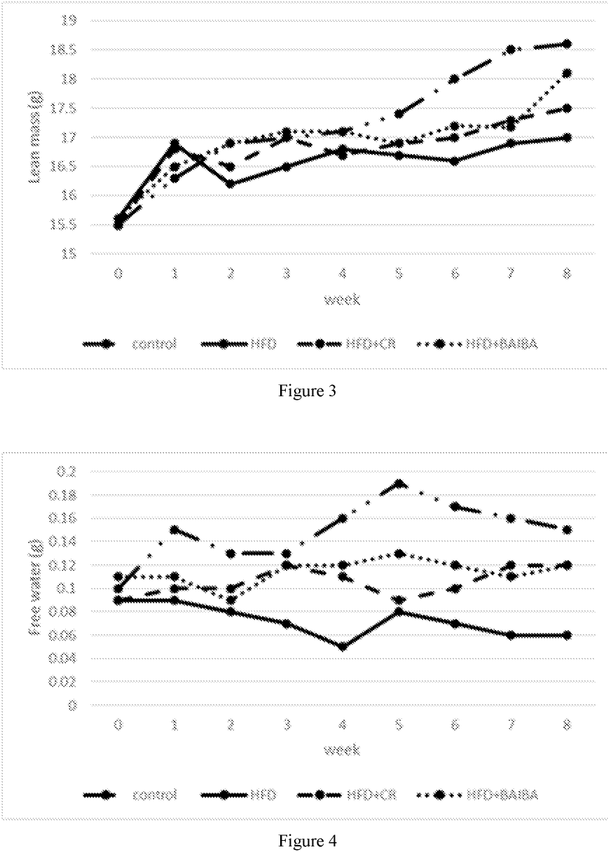

FIG. 3 is a graph of the lean mass changes of four groups of mice during 8 weeks of feeding intervention. As shown in FIG. 3, the lean mass of mice in group B (HFD) was significantly less than that in group A (control); the lean mass curves of group C (HFD+CR) and group D (HFD+BAIBA) were very similar, by the 8th week, the lean mass of group D (HFD+BAIBA) was significantly more than that of group C (HFD+CR). Group C achieved caloric restriction through a restricted HFD diet, but in a manner that is likely to lead to malnutrition and health risks; group D, fed a high-fat diet with BAIBA supplementation, mimicked the lean-mass-increasing effect of caloric restriction without causing malnutrition.

FIG. 4 is a graph of the free water changes of four groups of mice during 8 weeks of feeding intervention. As shown in FIG. 4, after the beginning of the intervention, the free water content of group B (HFD) mice was always significantly lower than that of group A (control); the free water contents of group C (HFD+CR) and group D (HFD+BAIBA) were higher or lower compared to each other, but both were significantly higher than that in group B (HFD). Group C achieved caloric restriction through a restricted HFD diet, but in a manner that is likely to lead to malnutrition and health risks; group D, fed a high-fat diet with BAIBA supplementation, mimicked the body-water-content-increasing and body-composition-improving effect of caloric restriction without causing malnutrition.

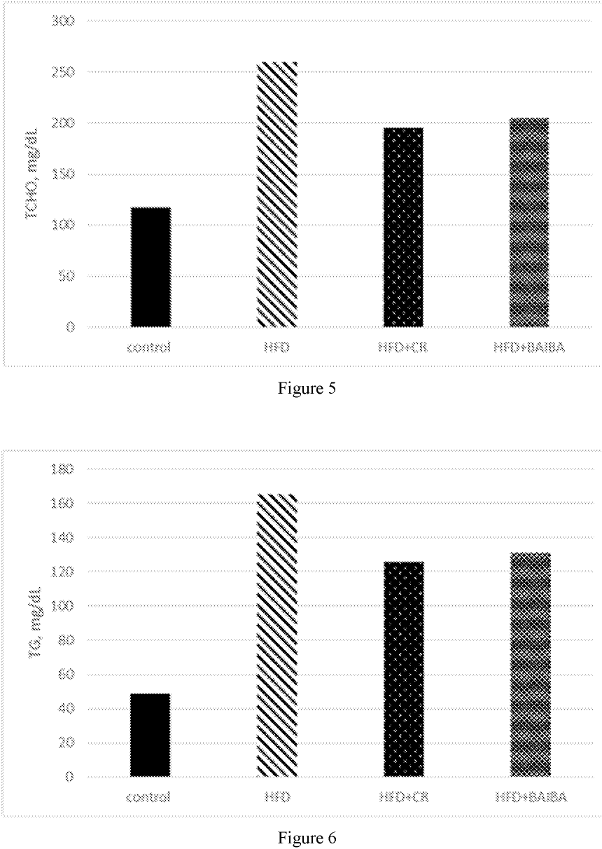

FIG. 5 is a graph comparing total cholesterol (TCHO) levels of four groups of mice measured after 8 weeks of treatment. As shown in FIG. 5, the TCHO level of mice in group B (HFD) was significantly higher than that in group A (control); group C (HFD+CR) achieved calorie restriction through a restricted HFD diet, which could significantly decrease TCHO level, but the way of calorie restriction is likely to lead to malnutrition and cause health risks; the TCHO level of group D (HFD+BAIBA) was also significantly lower than that of group B (HFD) and close to that of group C, indicating that BAIBA supplementation while feeding a high-fat diet can mimic the cholesterol-lowering effect of caloric restriction without causing malnutrition.

FIG. 6 is a graph comparing triglyceride (TG) levels of four groups of mice measured after 8 weeks of treatment. As shown in FIG. 6, the TG level of mice in group B (HFD) was significantly higher than that in group A (control); group C (HFD+CR) achieved calorie restriction through a restricted HFD diet, which could significantly decrease TG level, but the way of calorie restriction is likely to lead to malnutrition and cause health risks; the TG level of group D (HFD+BAIBA) was also significantly lower than that of group B (HFD) and close to that of group C, indicating that BAIBA supplementation while feeding a high-fat diet can mimic the triglyceride-lowering effect of caloric restriction without causing malnutrition.

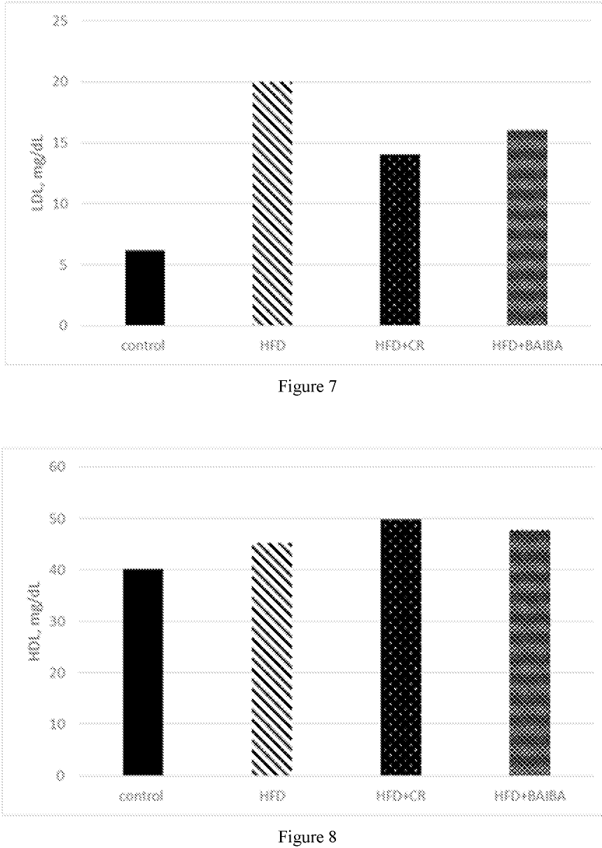

FIG. 7 is a graph comparing low-density lipoprotein (LDL) levels of four groups of mice measured after 8 weeks of treatment. As shown in FIG. 7, the LDL level of mice in group B (HFD) was significantly higher than that in group A (control); group C (HFD+CR) achieved calorie restriction through a restricted HFD diet, which could significantly decrease LDL level, but the way of calorie restriction is likely to lead to malnutrition and cause health risks; the LDL level of group D (HFD+BAIBA) was also significantly lower than that of group B (HFD), indicating that BAIBA supplementation while feeding a high-fat diet can mimic the LDL-lowering effect of caloric restriction without causing malnutrition.

FIG. 8 is a graph comparing high-density lipoprotein (HDL) levels of four groups of mice measured after 8 weeks of treatment. As shown in FIG. 8, the HDL levels of four groups were not significantly different, but both groups C (HFD+CR) and D (HFD+BAIBA) had higher HDL levles compared to group B (HFD). In group C, calorie restriction through a restricted HFD diet could increase LDL levels, but the way of calorie restriction is likely to lead to malnutrition and pose health risks; Group D, fed a high-fat diet with BAIBA supplementation, mimicked the HDL-enhancing effect of caloric restriction without causing malnutrition.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### OTHER EMBODIMENTS

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A method for mimicking one or more biological benefits of caloric restriction in a mammal, comprising administrating to the mammal an effective amount of β-aminoisobutyric acid, or an analog, metabolite or derivative thereof, or a pharmaceutically acceptable salt, ester, acid, polymer thereof.

2. The method of claim 1, wherein the one or more biological benefits comprise: lowering heart rate, blood pressure, low-density lipoprotein, cholesterol and triglycerides; raising high-density lipoprotein; improving insulin sensitivity and normalizing blood glucose; maintaining DNA integrity; reducing oxidative stress; decreasing body temperature; reducing body fat mass, including visceral obesity, while increasing muscle mass; increasing lean mass; improving body composition; losing weight; enhancing the ability to engage in sports activities; improving brain function, including memory, cognition, and mood; or stimulating growth factors.

3. The method of claim 1, wherein the β-aminoisobutyric acid is of configuration L or D or a form of a mixture of L and D configurations.

4. The method of claim 1, wherein the β-aminoisobutyric acid is administrated in an amount ranging from 0.1 mg/day-5000 mg/day.

5. The method of claim 1, wherein the β-aminoisobutyric acid is administrated in an amount ranging from 100 mg/day-2500 mg/day.

6. The method of claim 1, wherein the β-aminoisobutyric acid is administrated in a form of aqueous solution, aqueous suspension, capsule, drop, granule, liquid, powder, syrup, tablet, functionalized food, beverage, toothpaste, or sublingual articles.

7. The method of claim 1, wherein the mammal is human.

8. The method of claim 1, wherein the β-aminoisobutyric acid is administrated orally, by intravenous injection, by intramuscular injection, intraperitoneally or sublingually.

9. The method of claim 1, wherein the β-aminoisobutyric acid is prepared in a form of nutritional, drinking, or pharmaceutical composition, for use in a food, drink, nutritional, or pharmaceutical products.

10. The method of claim 1, wherein the β-aminoisobutyric acid is administrated as a dietary supplement or an ingredient in a food.

11. A composition capable of mimicking one or more biological benefits of caloric restriction in a mammal, comprising an effective amount of β-aminoisobutyric acid, an analog, metabolite or derivative thereof, or a pharmaceutically acceptable salt, ester, acid, polymer thereof.

12. The composition of claim 11, wherein the one or more biological benefits comprise: lowering heart rate, blood pressure, low-density lipoprotein, cholesterol and triglycerides; raising high-density lipoprotein; improving insulin sensitivity and normalizing blood glucose; maintaining DNA integrity; reducing oxidative stress; decreasing body temperature; reducing body fat mass, including visceral obesity, while increasing muscle mass; increasing lean mass; improving body composition; losing weight; enhancing the ability to engage in sports activities; improving brain function, including memory, cognition, and mood; or stimulating growth factors.

13. The composition of claim 11, wherein the β-aminoisobutyric acid is administrated in an amount ranging from 0.1 mg/day-5000 mg/day.

14. The composition of claim 11, wherein the β-aminoisobutyric acid is administrated in an amount ranging from 100 mg/day-2500 mg/day.

15. The composition of claim 11, wherein the β-aminoisobutyric acid is administrated in a form of aqueous solution, aqueous suspension, capsule, drop, granule, liquid, powder, syrup, tablet, functionalized food, beverage, toothpaste, or sublingual articles.

16. The composition of claim 11, wherein the β-aminoisobutyric acid is administrated orally, by intravenous injection, by intramuscular injection, intraperitoneally or sublingually.

17. The composition of claim 11, wherein the β-aminoisobutyric acid is prepared in a form of nutritional, drinking, or pharmaceutical composition, for use in a food, drink, nutritional, or pharmaceutical products.

18. The composition of claim 11, wherein the composition is a dietary composition or supplement.

19. Use of β-aminoisobutyric acid in manufacturing a composition capable of mimicking one or more biological benefits of caloric restriction in a mammal.

20. The use of claim 19, wherein the one or more biological benefits comprise: lowering heart rate, blood pressure, low-density lipoprotein, cholesterol and triglycerides; raising high-density lipoprotein; improving insulin sensitivity and normalizing blood glucose; maintaining DNA integrity; reducing oxidative stress; decreasing body temperature; reducing body fat mass, including visceral obesity, while increasing muscle mass; increasing lean mass; improving body composition; losing weight; enhancing the ability to engage in sports activities; improving brain function, including memory, cognition, and mood; or stimulating growth factors.
